(19) United States
(12) Patent Application Publication
(10) Pub. No.: US 2021/0398450 A1
(43) Pub. Date: Dec. 23, 2021

(54) METHOD OF IMPROVING SELF-ESTEEM, CONFIDENCE, AND PUBLIC SPEAKING SKILLS

(71) Applicant: Chizoma Nosiri, Crofton, MD (US)

(72) Inventor: Chizoma Nosiri, Crofton, MD (US)

(21) Appl. No.: 16/906,819

(22) Filed: Jun. 19, 2020

(51) Int. Cl. G09B 19/04; A61B 5/16; G16H 20/70

(52) U.S. Cl. CPC G09B 19/04; G16H 20/70; A61B 5/165; A61B 5/167

(57) ABSTRACT

A method of improving self-esteem, confidence, and public speaking skills of an individual, including asking the individual a series of questions regarding a plurality of social concerns of the individual, rating a level of self-esteem, a level of confidence, and a level of public speaking skills of the individual, identifying and assessing the social concerns of the individual as related to self-esteem, confidence, and public speaking skills, using a diagram having a plurality of terms regarding an intra-personal point-of-view and an inter-personal point-of-view, asking the individual to identify the terms from the diagram that form a root cause of the social concerns of the individual, and assisting the individual to identify at least one additional area of concern based on the rating of the level of self-esteem, the level of confidence, and the level of public speaking skills of the individual.

### BACKGROUND OF THE INVENTION

### 1. Field of the Invention

The present general inventive concept relates to a method of improving social and mental well-being, and more particularly, to a method of improving self-esteem, confidence, and public speaking skills of an individual by identifying issues controlled by the individual and issues controlled by another person.

### 2. Background

Many methods exist to improve one's social and mental well-being. Various techniques include questionnaires to score and determine one's baseline self-esteem and confidence, providing a personalized booster for one's self-esteem, and rewiring one's view of oneself via activities designed to guide changes to one's way of thinking.

The utilization of such techniques is well-known. However, such techniques do not determine root causes of underlying problems with respect to one's self-esteem, confidence, and public speaking skills by comparing one's intra-personal, or inner, point-of-view to one's inter-personal, or outer, point-of-view. Specifically, the existing methods do not train a person to focus more on issues controlled by the person, and less on issues not controlled by the person.

Therefore, what is needed is a method of improving self-esteem, confidence, and public speaking skills of an individual by identifying issues controlled by the individual and issues controlled by another person, and redirecting focus towards the issues controlled by the individual.

### SUMMARY OF THE INVENTION

Certain of the foregoing and related aspects are readily attained according to the present general inventive concept by providing a method of improving self-esteem, confidence, and public speaking skills of an individual, including asking the individual a series of questions regarding a plurality of social concerns of the individual, rating a level of self-esteem, a level of confidence, and a level of public speaking skills of the individual, identifying and assessing the social concerns of the individual as related to self-esteem, confidence, and public speaking skills, using a diagram having a plurality of terms regarding an intra-personal point-of-view and an inter-personal point-of-view, asking the individual to identify the terms from the diagram that form a root cause of the social concerns of the individual, and assisting the individual to identify at least one additional area of concern based on the rating of the level of self-esteem, the level of confidence, and the level of public speaking skills of the individual.

The diagram includes a first set of terms relating to self-esteem and confidence for the intra-personal point-of-view, and a second set of terms relating to presentation and performance for the inter-personal point-of-view.

The level of self-esteem, the level of confidence, and the level of public speaking skills are each given a score on a scale of 0 to 10. A score between 8 and 10 indicates a healthy level.

The step of asking the individual the series of questions may involve using a deck of cards to ask the questions.

Certain of the foregoing and related aspects are readily attained according to the present general inventive concept by also providing a method of improving public speaking skills of an individual, including identifying, in a first set of categories, a plurality of issues controlled by the individual, and, in a second set of categories, a plurality of issues controlled by another person while listening to the individual speak, focusing on the issues controlled by the individual in a greater relation to the issues controlled by the other person, and testing control of the issues by the individual via public speaking, to determine a level of self-esteem, a level of confidence, and a level of public speaking skills of the individual.

In contrast with conventional methods disclosed by the prior art, the present general inventive concept utilizes a comparison of parameters from an intra-personal point-of-view and parameters from an inter-personal point-of-view to assist an individual to improve their self-esteem, confidence, and public speaking skills. Specifically, one focuses more on issues that one controls, and less on issues that others control.

### BRIEF DESCRIPTION OF THE DRAWINGS

These and/or other aspects of the present general inventive concept will become apparent and more readily appreciated from the following description of the embodiments, taken in conjunction with the accompanying drawings of which:

FIG. 1 illustrates a flowchart of a method of improving self-esteem, confidence, and public speaking skills of an individual.

FIG. 2 is a plan view of a card used to ask questions, provide phrases or statements to build self-esteem, confidence, and public speaking skills in the method of FIG. 1.

FIG. 3 is a schematic of a diagram implemented in the method of FIG. 1.

FIG. 4 illustrates a flow chart of a method of improving public speaking skills of an individual.

FIG. 5 is a schematic of a first set of categories and a second set of categories implemented in the method of FIG. 4.

FIG. 6 is a schematic designed to aid an individual to achieve a spiritual view of themselves.

FIG. 7 illustrates a flow chart of further steps of the method of FIG. 4.

FIG. 8 is a schematic of a plan for preparing to give a presentation.

FIG. 9 is a schematic diagram depicting Speaker's Gym session workshop class implementations of the method illustrated in FIG. 1 and the method illustrated in FIGS. 4 and 7.

FIG. 10 is a plan view of a game used to execute the method of FIG. 1 and the method of FIGS. 4 and 7.

FIG. 11 is a schematic depicting variants of virtual implementations of the method of FIG. 1 and the method of FIGS. 4 and 7.

### DESCRIPTION OF THE PREFERRED EMBODIMENTS

The present inventive concept will now be described more fully with reference to the accompanying drawings, in which exemplary embodiments of the present general inventive concept are illustrated. The inventive concept may, however, be embodied in many different forms and should not be construed as being limited to the embodiments set forth herein; rather, these embodiments are provided so that this disclosure will be thorough and complete, and will fully convey the concept of the invention to those skilled in the art. Like reference numerals refer to like elements throughout.

The present general inventive concept includes a method of improving self-esteem, confidence, and public speaking skills.

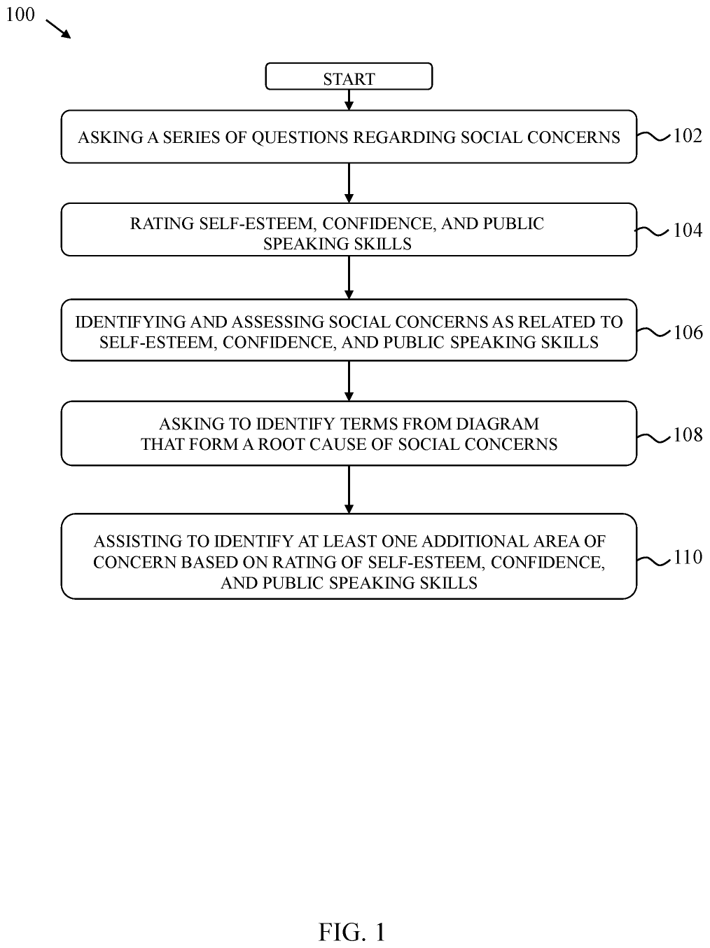

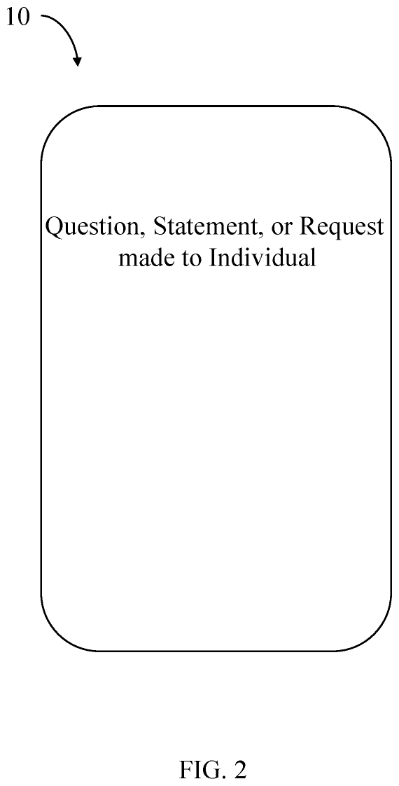

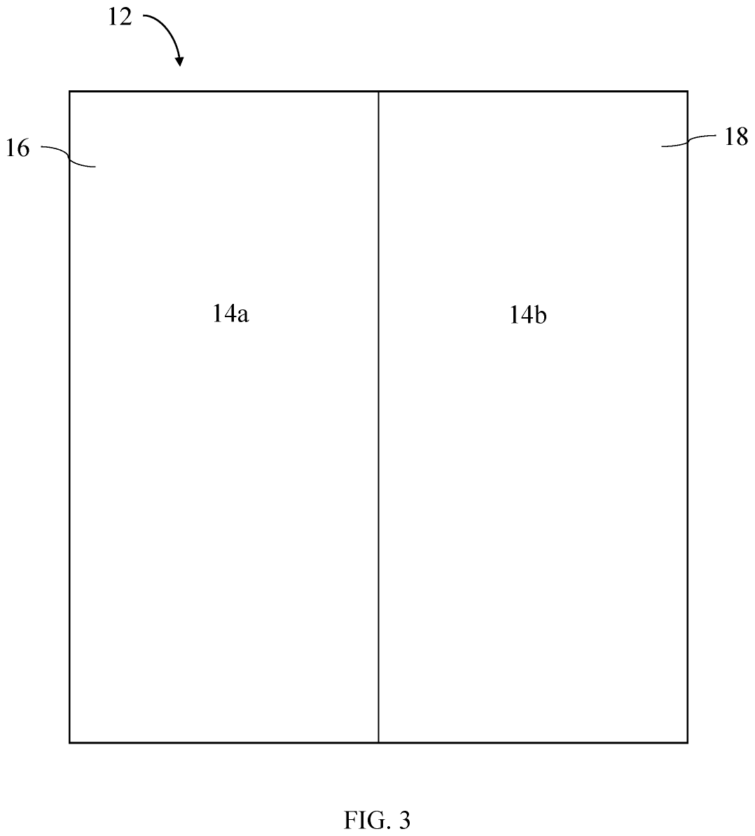

FIG. 1 illustrates a flow chart of an exemplary method 100 of improving self-esteem, confidence, and public speaking skills of an individual, according to the present general inventive concept. FIG. 2 is a plan view of a card used to ask questions, provide phrases or statements to build self-esteem, confidence, and public speaking in the method of FIG. 1. FIG. 3 is a schematic of a diagram implemented in the method of FIG. 1.

In the present embodiment, the method 100 includes asking the individual a series of questions regarding a plurality of social concerns of the individual, at step 102. Non-limiting examples of social concerns that can cause anxiety in an individual include ageism, sexism, ableism, racism, classism, and social or professional positions and titles. In an exemplary embodiment, step 102 is accomplished by using a deck of cards 10 to ask the questions. An example of such a card 10 is depicted in FIG. 2. Such cards 10 can be used, for instance, if the method 100 is played as a game. The questions can also be provided in a book (e.g. a self-help book or a guidebook for the method 100). In one instance, the cards 10 also contain statements of positive affirmation, for instance, requesting the individual to state one of their personal strengths. Examples of such personal strengths include gifts, talents, and skills, discussed further below. In another instance, the cards 10 also include requests for the individual to describe aspects of their behavior and personality. An example is asking the individual to describe their personal ability to control how much energy they devote to positive thoughts and interactions versus negative thoughts and interactions.

Next, at step 104, each of a level of self-esteem, a level of confidence, and a level of public speaking skills of the individual are rated. In an embodiment, the level of self-esteem, the level of confidence, and the level of public speaking skills are each given a score on a scale of 0 to 10. A score between 8 and 10 indicates a healthy level. The individual also explains why they chose these scores.

Self-esteem refers to how one values and accepts oneself, and should not change based on a given environment or situation. If an individual does change their self-esteem based on environment or situation, then their self-esteem is poor. Self-esteem does not include being narcissistic or condescending towards others.

Confidence, or self-confidence, refers to one's belief in one's capabilities, such as setting and completing a goal.

Public speaking skills refer to one's ability to speak effectively to an audience, as well as one's confidence in one's ability to do so.

Next, at step 106, the social concerns of the individual are identified and assessed as related to self-esteem, confidence, and public speaking skills. Used in this step 106 is a diagram 12, illustrated in FIG. 3, that has a plurality of terms 14a, 14b regarding an intra-personal point-of-view 16 and an inter-personal point-of-view 18. In an embodiment, the diagram 12 includes a first set of terms 14a relating to self-esteem and confidence for the intra-personal, or inner, point-of-view 16. The intra-personal point-of-view 16 refers to dialogue that one has with oneself. Non-limiting examples of such terms include judgment, intimidation, comparison, self-doubt, self-lacking, spotlight anxiety, hesitation, inadequacy, inferiority, insecurity, timidity, fear of the unknown, self-unimportance, imperfections, negative self-perception, and lacking a voice. The diagram 12 also includes a second set of terms 14b relating to presentation and performance for the inter-personal, or outer, point-of-view 18. The inter-personal point-of-view 18 refers to communication that takes place between the individual and at least one other person. Non-limiting examples of such terms include eye contact, verbal messages, body language, appearance, gestures, tone of voice, persuasiveness, engagement tools, posture, code of attire, facial expression, word selection, knowledge, vocal expression, space-fillers, and kinesics.

Next, at step 108, the individual is asked to identify the terms 14a, 14b from the diagram 12 that form a root cause of the social concerns of the individual.

Next, at step 110, the individual is assisted to identify at least one additional area of concern based on the rating of the level of self-esteem, the level of confidence, and the level of public speaking skills of the individual. Such areas of concern may be significant reasons as to why the individual is lacking in self-esteem, confidence, and public speaking skills.

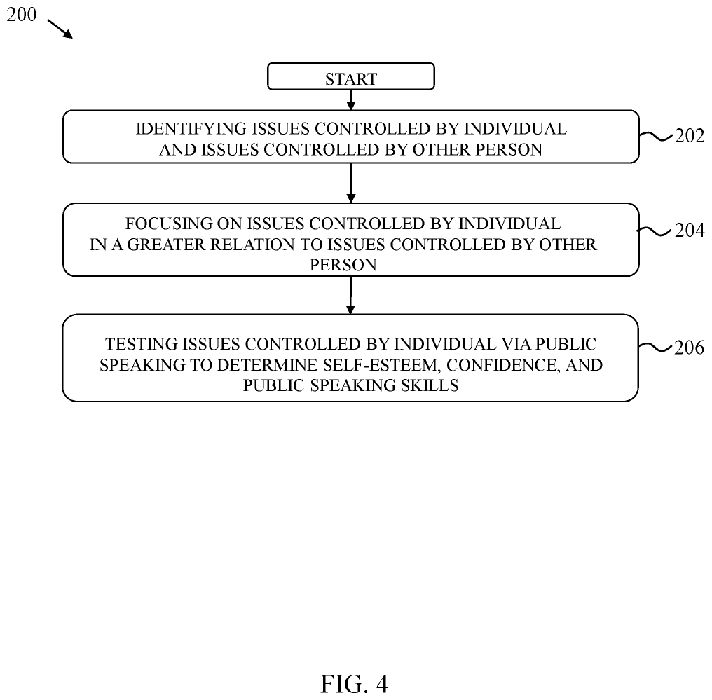

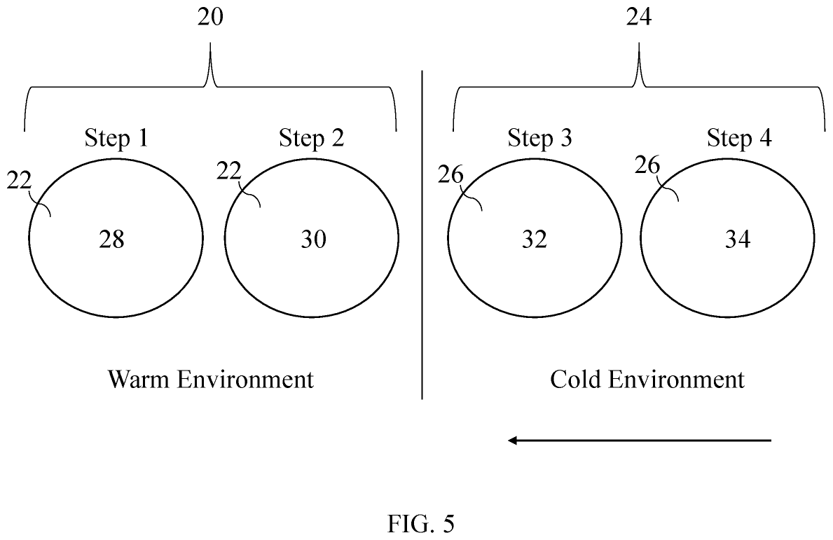

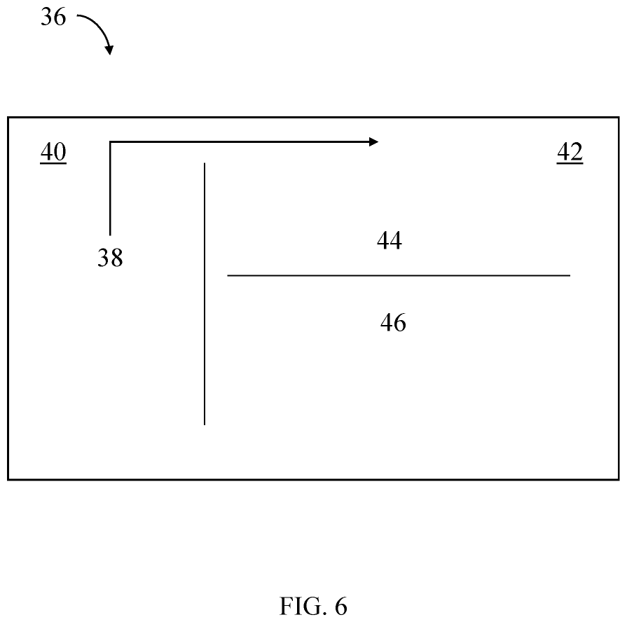

FIG. 4 illustrates a flow chart of an exemplary method 200 of improving public speaking skills of an individual. FIG. 5 is a schematic of a first set of categories and a second set of categories implemented in the method of FIG. 4. FIG. 6 is a schematic designed to aid an individual to achieve a spiritual view of themselves.

In the present embodiment, the method 200 includes identifying, in a first set of categories 20, a plurality of issues controlled by the individual 22, and, in a second set of categories 24, a plurality of issues controlled by another person 26 while listening to the individual speak, at step 202. In an exemplary embodiment, the first set of categories 20 includes a plurality of traits of the individual 28 and a plurality of traits intended by a presentation given by the individual 30. Non-limiting examples of such traits 28, 30 include the individual as a speaker and content of the presentation. The issues controlled by the individual 22 include, for instance, gifts, talents, skills, and content of the presentation given by the individual. Gifts refer to innate qualities. Talents refer to a learned ability that has become easy to perform. Skills refer to a learned ability that the individual has not necessarily fully mastered. As depicted in FIG. 5, the first set of categories 20 is presented as Step 1 and Step 2. The second set of categories 24 includes a plurality of traits of the other person 32 and a plurality of traits received by the other person from the presentation given by the individual 34. Non-limiting examples of such traits 32, 34 include the other person as a listener and outcome of the presentation. The issues controlled by the other person 26 include, for instance, behavior, judgment, questions asked, comments, expectations, and impact of the presentation given by the individual on the other person. As depicted in FIG. 5, the second set of categories 24 is presented as Step 3 and Step 4. The issues 22, 26 in both the first set of categories 20 and the second set of categories 24 interact with each other in an environment or situation to drive a social dynamic between the individual and the other person. As further illustrated in FIG. 5, the issues controlled by the individual 22 create a warm environment for the individual, wherein the individual is comfortable with themselves if they focus on these issues 22. The issues controlled by the other person 26 create a cold environment for the individual, wherein the individual is uncomfortable with themselves if they focus on these issues 26. As indicated by the arrow in FIG. 5, the object is to redirect focus towards the issues controlled by the individual 22, to create a warm environment.

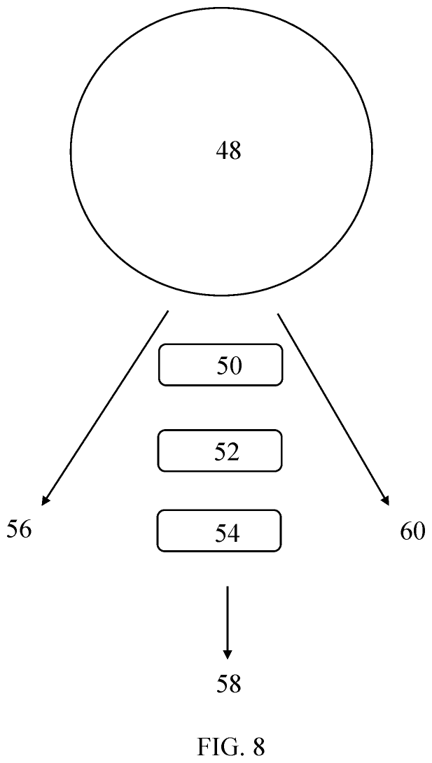

Next, at step 204, the issues controlled by the individual 22 are focused on in a greater relation to the issues controlled by the other person 26. In other words, when the individual focuses too much on the issues controlled by the other person 26, their intra-personal point-of-view 16 is overridden. Self-esteem, confidence, and public speaking skills therefore suffer. However, when the individual focuses more on the issues within their control 22, the inter-personal point-of-view 18 becomes of less importance, as the individual accepts that they cannot control the inter-personal point-of-view 18. Self-esteem, confidence, and public speaking skills therefore improve. Accepting what one does not control reduces one's need for approval from others and one's negative thoughts towards oneself. An example of focusing on the issues controlled by the individual 22, as depicted in FIG. 8, can be seen while preparing to give a presentation. The individual plans to speak 48 and must determine a topic 50 of the presentation, as well as content 52 and an intended message 54 to be conveyed. The individual must therefore focus on: understanding the topic 56, connecting to the topic 58 (i.e. the individual becomes connected to the topic), and designing the presentation for the topic 60.

Next, at step 206, control of the issues by the individual 22 is tested via public speaking, to determine a level of self-esteem, a level of confidence, and a level of public speaking skills of the individual. When the individual gives the presentation, they must not only provide and discuss the content 52, but must also critically examine the content 52 in a way that creates a critical discourse, or a thorough discussion of the content 52. In so doing, the individual can create a call to action, i.e. a take-away message that the audience gains from listening to the presentation. Such a call to action is possible when the issues controlled by the individual 22 are mastered, and the issues controlled by the other person 26 listening to the individual are let go of. The individual feels comfortable speaking and is not concerned with making mistakes or being judged or victimized. As discussed above, such a state is called a warm environment. If such a state is not achieved, the individual is in a cold environment.

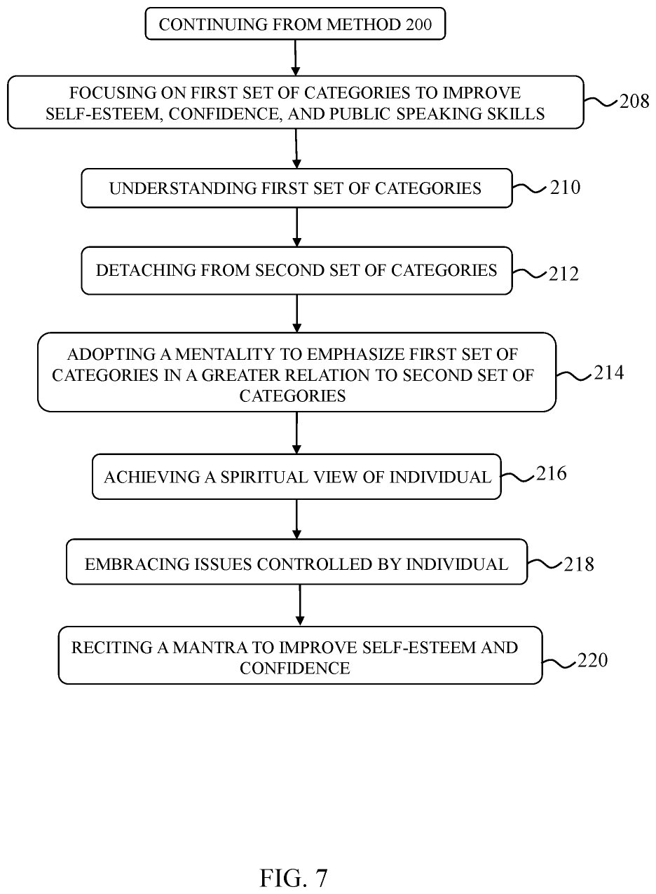

FIG. 7 illustrates a flow chart of further steps of the method 200 of improving public speaking skills of an individual and FIG. 8 is a schematic of a plan for preparing to give a presentation.

In an exemplary embodiment, the method 200 further includes focusing on the first set of categories 20 to improve self-esteem, confidence, and public speaking skills of the individual, at step 208.

In another exemplary embodiment, the method 200 further includes understanding the first set of categories 20, at step 210, and detaching from the second set of categories 24, at step 212.

In a further exemplary embodiment, the method 200 includes adopting a mentality in which the first set of categories 20 is emphasized in a greater relation to the second set of categories 26, at step 214, and achieving a spiritual view of the individual, at step 216 which is achieved through FIG. 7.

In yet another exemplary embodiment, the method 200 further includes embracing the issues controlled by the individual 22, at step 218, and reciting a mantra to improve self-esteem and confidence of the individual, at step 220. In a particular embodiment, the mantra focuses on a spiritual aspect of self-esteem and confidence. For example, the mantra may be as follows:
I AM the Speaker.
Speaking is my right and my voice will be heard.
I will focus on my Gifts, Talents, and Skills, for I was born to speak.
As the Speaker, my Topic is central, and I dwell in positive thoughts.
I AM in control of my mind.
I AM in control of my body.
I AM in control of my words.
I speak with Confidence.
I speak with Power.
I speak with Passion.
No matter what happens, I AM Greatness.
What I Believe I become.
I am Enough. I am Esteemed. I am Empowered.

By reciting such a mantra and focusing on the issues controlled by the individual 22, the individual comes to see themselves as a human, flawed, but also resilient and capable of self-improvement.

FIG. 6 illustrates a schematic 36 designed to aid an individual to achieve a spiritual view of themselves by separating their own innate worth 38 from the external value placed upon them by society. As indicated by the arrow in FIG. 6, the innate worth 38 is what one brings from a spiritual realm 40 and enters with into the physical world 42, including gifts, talents, and skills. This can be defined as one's own personal greatness or glory. With the innate worth 38, one goes through the journey 44 of life, undergoing events and experiencing emotions such as happiness and sadness. Throughout the journey 44, society puts a plurality of constraints 46 upon the individual that are made by humans and motivated by ego. Such constraints 46 include for instance culture, value systems, race, ethnicity, gender, professional position, and status. Such constraints 46 are meant to define the individual, and often confine them in the process. Thus, the individual becomes distracted and separated from their innate worth 38. The schematic 36 reminds the individual of this distraction and separation, and reminds them to instead separate from the constraints 46 and reunite with their innate worth 38. Thus, FIGS. 5 and 6, when used together, allow the individual to deconstruct their interaction with themselves and with others by separating the first set of categories 20 from the second set of categories 24, and focusing on the first set of categories 20 (which is derived from their innate worth 38) in a greater relation to the second set of categories 24 (which is created by the constraints 46).

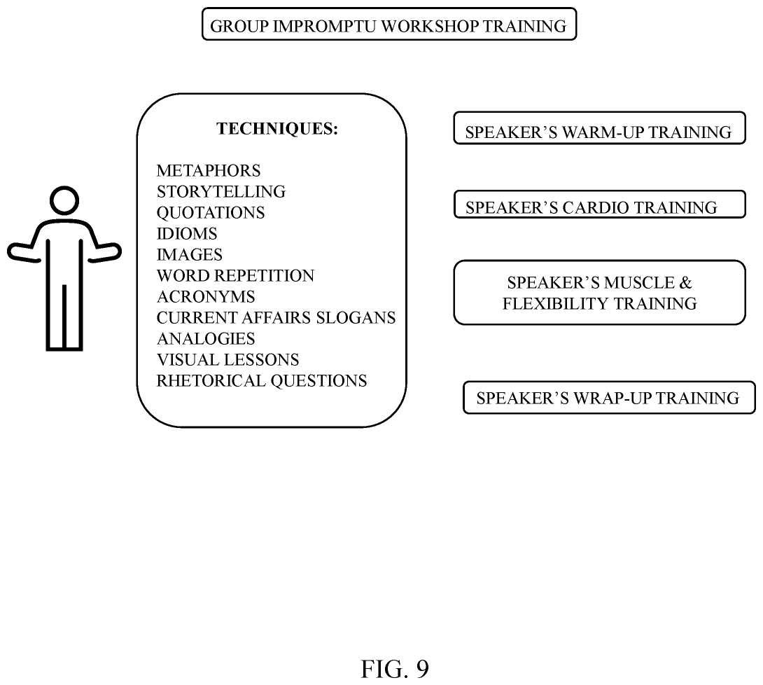

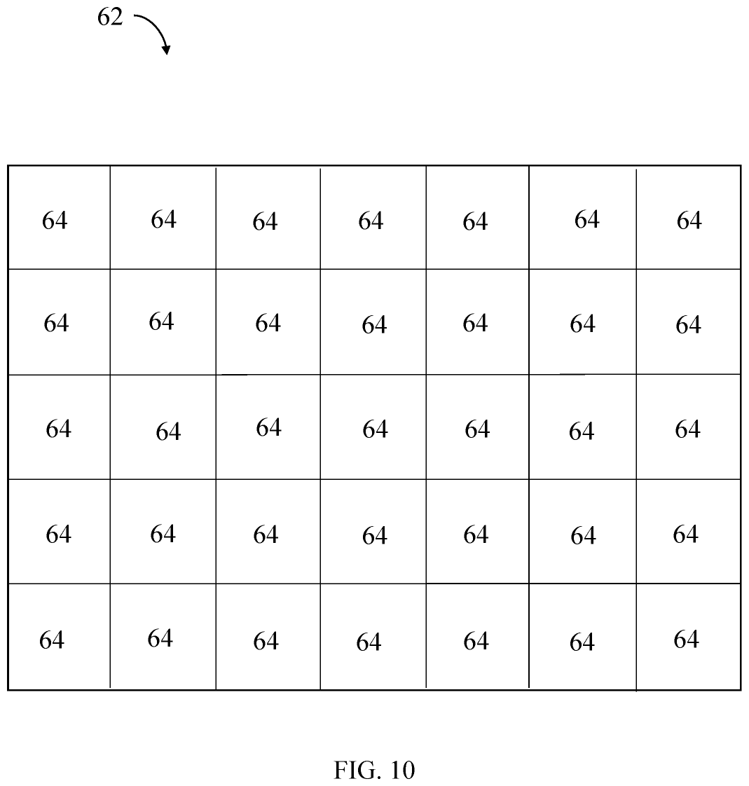

FIG. 9 is a schematic diagram depicting Speaker's Gym session workshop class implementations of the method illustrated in FIG. 1 and the method illustrated in FIGS. 4 and 7. FIG. 10 is a plan view of a game used to execute the method of FIG. 1 and the method of FIGS. 4 and 7 and FIG. 11 is a schematic depicting variants of virtual implementations of the method of FIG. 1 and the method of FIGS. 4 and 7.

As depicted in FIG. 9, the above methods 100, 200 can be conducted in person or virtually through the designed Speaker's Gym group workshop class sessions. As illustrated, the Speaker's Gym is designed as a public speaking technique group workshop class session platform dedicated to allowing individuals to build self-esteem, confidence, and public speaking skills in volunteer lead group format. The Speaker's Gym weekly sessions are structured as impromptu public speaking training with warm-up, cardio, muscle & flexibility, and wrap up tasks using Presentation Techniques: metaphors, images, current affairs, words repetition, acronyms, slogans, analogies, idioms, storytelling, object lessons, quotations, rhetorical questions, and more) to enhance methods 100, 200.

The above methods 100, 200 can be conducted in person. For instance, a friend, a relative, a teacher, a coach or a counselor can guide the individual through the methods 100, 200. The other person listening to the individual speak can be a single person or a group of people in the same room as the individual. The methods 100, 200 can also be conducted via games. An example of such a game is depicted in FIG. 10, as a bingo game 62 containing a plurality of terms 64 that the individual identifies as part of their social concerns, the intra-personal point-of-view 16, gifts, talents, skills, and their innate worth 52.

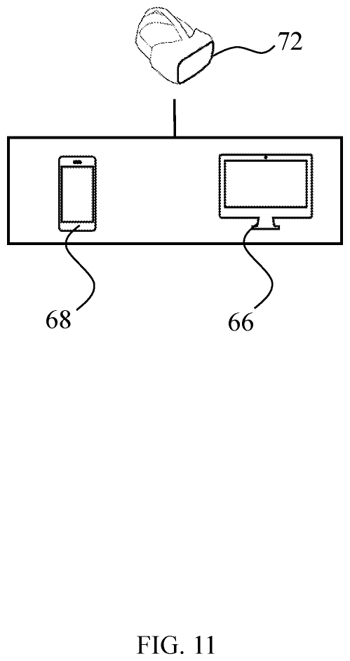

As depicted in FIG. 11, the above methods 100, 200 can also be conducted virtually. Non-limiting examples of virtual (or non-in-person) options include the use of a website and/or a web application via a computer 66, a phone call via a mobile telephone 68, a mobile application via a mobile phone 68, and a virtual reality headset 72. Any of these options may include the use of a virtual/augmented reality headset 72. Such applications have features that include, but are not limited to, video and audio tutorials (regarding, for instance, intra-personal point-of-view 16 and inter-personal point-of-view 18, self-esteem, innate worth 52, confidence, and self-empowerment), connecting to a topic and an audience, and sharing links to the video and audio tutorials. In one instance, the steps 102-110 and 202-204 may be completed by the individual filling out a questionnaire. In another instance, the steps 102-110 and 202-204 may be completed via a telephone call, or virtual interaction between the individual and an agent (e.g. a coach or counselor) using a website, a web application, or a mobile application. The steps 206-220 may also be completed via a virtual interaction between the individual, an agent, an audience (i.e. another person listening to the individual speak) using similar implementation.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

What is claimed is:

1. A method of improving self-esteem, confidence, and public speaking skills of an individual, comprising the steps of:
asking the individual a series of questions regarding a plurality of social concerns of the individual;
rating a level of self-esteem, a level of confidence, and a level of public speaking skills of the individual;
identifying and assessing the social concerns of the individual as related to self-esteem, confidence, and public speaking skills, using a diagram having a plurality of terms regarding an intra-personal point-of-view and an inter-personal point-of-view;
asking the individual to identify the terms from the diagram that form a root cause of the social concerns of the individual; and
assisting the individual to identify at least one additional area of concern based on the rating of the level of self-esteem, the level of confidence, and the level of public speaking skills of the individual.

2. The method of claim 1, wherein the diagram includes a first set of terms relating to self-esteem and confidence for the intra-personal point-of-view, and a second set of terms relating to presentation and performance for the inter-personal point-of-view.

3. The method of claim 1, wherein:
the level of self-esteem, the level of confidence, and the level of public speaking skills are each given a score on a scale of 0 to 10.

4. The method of claim 3, wherein a score between 8 and 10 indicates a healthy level.

5. The method of claim 1, wherein the step of asking the individual the series of questions involves using a deck of cards to ask the questions.

6. A method of improving public speaking skills of an individual, comprising the steps of:
identifying, in a first set of categories, a plurality of issues controlled by the individual, and, in a second set of categories, a plurality of issues controlled by another person while listening to the individual speak;
focusing on the issues controlled by the individual in a greater relation to the issues controlled by the other person; and
testing control of the issues by the individual via public speaking, to determine a level of self-esteem, a level of confidence, and a level of public speaking skills of the individual.

7. The method of claim 6, wherein:
the first set of categories comprises a plurality of traits of the individual and a plurality of traits intended by a presentation given by the individual; and
the second set of categories comprises a plurality of traits of the other person and a plurality of traits received by the other person from the presentation given by the individual.

8. The method of claim 7, wherein:
the issues controlled by the individual comprise gifts, talents, skills, and content of the presentation given by the individual; and
the issues controlled by the other person comprise behavior, judgment, questions asked, comments, expectations, and impact of the presentation given by the individual on the other person.

9. The method of claim 6, further comprising the step of focusing on the first set of categories to improve self-esteem, confidence, and public speaking skills of the individual.

10. The method of claim 9, further comprising the steps of:
understanding the first set of categories; and
detaching from the second set of categories.

11. The method of claim 10, further comprising the steps of:
adopting a mentality in which the first set of categories is emphasized in a greater relation to the second set of categories; and
achieving a spiritual view of the individual.

12. The method of claim 11, further comprising the steps of:
embracing the issues controlled by the individual; and
reciting a mantra to improve self-esteem and confidence of the individual.

13. The method of claim 12, wherein the mantra focuses on a spiritual aspect of self-esteem and confidence.
